SEQUENCE LISTING (4 sequences)

SEQ ID NO: 1 - protein, 21 aa, Artificial Sequence
  Synthetically prepared peptide sequence
VKTQATSREEPPRLPSKHRPG

SEQ ID NO: 2 - protein, 14 aa, Artificial Sequence
  Synthetically prepared peptide sequence
MDHGKYRQKQATPG

SEQ ID NO: 3 - protein, 2 aa, Artificial Sequence
  Synthetically prepared peptide sequence
QA

SEQ ID NO: 4 - protein, 252 aa, Artificial Sequence
  Synthetically prepared polypeptide sequence
QATSGSERMGHQSGTVHPGKTQATSGSERMGHQSGTVHPGKTQATSGSERMGHQSGTVHPGKTQATSGSERMGHQSGTVHPGKTQATSGSERMGHQSGTVHPGKTQATSGSERMGHQSGTVHPGKTQATSGSERMGHQSGTVHPGKTSKTSLGQSGASLQGSEKLTNGSKTSLGQSGASLQGSEKLTNGSKTSLGQSGASLQGSEKLTNGSKTSLGQSGASLQGSEKLTNGSKTSLGQSGASLQGSEKLTNG

(19) United States
(12) Patent Application Publication
(10) Pub. No.: US 2006/0240567 A1
(43) Pub. Date: Oct. 26, 2006

(54) Method of immobilizing a protein to a zeolite

(76) Inventors: Stanley Edward Brown, Copenhagen K (DK); Rune Wendelbo, Oslo (NO); Sanne Nygaard, Bronshoj (DK)

Correspondence Address: KNOBBE MARTENS OLSON & BEAR LLP, 2040 MAIN STREET, FOURTEENTH FLOOR, IRVINE, CA, 92614, US

(21) Appl. No.: 10/534,162

(22) PCT Filed: Nov. 7, 2003
(86) PCT No.: PCT/DK03/00771

§ 371 Date: May 19, 2006

(30) Foreign Application Priority Data
Nov. 8, 2002 (DK) PA 2002 01726

(51) Int. Cl. G01N 33/558

(52) U.S. Cl. 436/514; 435/325

(57) ABSTRACT

The present invention relates to a polypeptide tag and a method using said polypeptide tag sequence for immobilizing a protein on a microporous material, said microporous material is selected from the group consisting of zeolite or similar solid surfaces whereby loss of activity of said protein is less than 10% of the initial activity prior to immobilization, the method comprising the steps of: 1. Selecting a polypeptide tag capable of binding to the surface, 2. Immobilizing said protein by the steps of: attaching said polypeptide tag to the protein, and binding said polypeptide tag to the solid surface where step (a) and (b) is performed simultaneously or sequentially and when performed sequentially, the order of step (a) and (b) is random, subject to the limitation that the polypeptide tag does not consist only of histidine residues.

### FIELD OF INVENTION

The present invention relates in general to the field of immobilising proteins to a solid surface. In particular, novel polypeptide tags capable of distinguishing solid surfaces having identical atomic compositions and varying only in the spatial orientation of surface atoms with the objective of immobilising proteins to the solid surface and use thereof is provided.

### TECHNICAL BACKGROUND AND PRIOR ART

Proteins are the chemical building blocks from which cells, organs and tissues like muscle are made and on which nearly every biological reactions depends. Proteins also act as hormones, enzymes, and antibodies, which assist the body in combating invading germs. Proteins are made of long chains of even smaller building blocks of amino acids. The content of amino acids determines the size, structure, and length of the final protein molecule.

Thus, proteins play a crucial role in virtually all biological processes and some of their significant and remarkable activities can be explained as follows:
1. Enzymatic activity. Nearly all of the chemical reactions in biological systems are catalysed by enzymes which exhibit enormous catalytic power whereby the reaction rates are increased by at least a million-fold.
2. Transport and storage. Specific proteins transport many small molecules and ions from one place to another.
3. Coordinated motion. Two types of protein filaments may provide a sliding motion in order to introduce a motion. This is indicated by e.g. muscle contraction or by the propulsion of flagella of sperm cells.
4. Immune protection. Antibodies are highly specific binding proteins that recognise and combine with foreign substances such as viruses, bacteria and cells from other organisms. Thus, proteins are able to distinguish very specific features of substances, biological and non-biological.
5. Generation and transmission of impulses. The response of nerve cells to a specific stimulus is mediated and transmitted by protein receptors that can be triggered by specific small molecules, i.e. acetylcholine.
6. Control of growth and differentiation. Controlled sequential expression of genetic information is essential for the orderly growth ad differentiation of cells.

Thus, proteins can be useful for a large range of different applications, but common for all the applications is that the reaction is based on distinguishing or specifically recognising specific parts of a molecule or composition.

As mentioned, enzymes constitute one particular type of proteins capable of catalysing reactions with high specificity, and often a high catalytic efficiency and rate enhancement exceeding 100,000 fold are common. Enzymes are frequently used in many processes in industrial manufacturing, in the laboratory and in the home.

A subclass of enzymes is the proteolytic enzymes which are able to hydrolyse peptide bonds. Furthermore, some proteolytic enzymes are discriminating in their reactivity whereas others are quite undiscriminating.

Often, after an enzymatic reaction is conducted, it is desirable to remove the enzyme either to ease purification of the end product or to avoid interference with subsequent reactions. Proteolytic enzymes are commonly used in many processes but the enzymes are generally unstable in solution due to exposure to proteolytic activity.

In order to overcome this problem of unstable enzymes, immobilisation of the enzyme on a solid support facilitates removal and prevents the proteolytic enzymes from hydrolysing each other and the active lifetime is dramatically extended.

Conventional methods of immobilising enzymes do not control the orientation of the enzyme. Thus, only a fraction of the enzymes have their active sites exposed to a solvent causing a reduction in catalytic activity. Moreover, many methods of enzyme immobilisation constrain the flexibility of the enzyme and thus reduce catalytic activity.

The immobilisation of the protein to a solid surface can be performed by various procedures. One of these procedures includes the association of a protein with a polypeptide tag.

One strategy of associating or attaching the polypeptide tag to the protein can be by recombinant DNA procedures or by chemical modification. S. Brown (1992) reported the ability of Escherichia coli to bind specifically to iron oxide and not to other metal oxides, after being genetically modified. Concatamers of random oligonucleotides were introduced into a portion of the plasmid borne lamB gene encoding an external domain of the phage λ receptor. The experiments showed that sequences of oligonucleotides were able to recognise specific solid surfaces.

S. Brown (1997) discloses that repeating polypeptide sequences are able to bind to gold surfaces. The experiment showed that the avidity of the binding polypeptide to bind with gold was dependent on the number of polypeptide repeats and the presence of salt. Thus, it was concluded that the binding between the protein and gold was caused by the repeating polypeptide sequence, as only few native proteins are able to adhere to gold, and the binding activity was dependent on the number of repeating polypeptide sequences.

Another strategy of attaching a protein tag onto a protein is by chemical modification i.e. by disulphide bonding between SH-containing residues (such as cysteine) of the protein with SH-containing residues of the protein tag. This strategy can only be applied to those proteins in whichn the existence of SH-containing residues can be controlled.

The ability of proteins or polypeptide tags to distinguish subtle differences between biological molecules is widely described in the prior art, but the degree to which proteins can distinguish differences between solid surfaces remains partly an unanswered question.

This means that the ability of proteins to distinguish surfaces of metals, metal oxides, metal carbonates and semiconductors that vary in atomic composition is well documented. However, the ability to distinguish solid surfaces having identical atomic compositions and varying only in the spatial orientation of surface atoms is apparently rare among described proteins.

C. A. Knight et al. (1991) and S. P. Graether et al. (2000) disclose that selected antifreeze proteins bind selectively to only one of the exposed planes of ice, whereas others bind to all exposed planes of ice. Ice is not a suitable solid surface for the immobilisation of proteins which should be used in the purification of an analyte because of the requirement of very cold temperatures to avoid defrosting of the solid surface.

Perhaps the differences in surface charge and atomic composition on exposed planes of such common biological crystallites as calcite alleviates the evolutionary need for such subtle discrimination and a greater understanding of the ability of proteins to distinguish solid surfaces may open new disciplines in engineering, particularly at nanometer length scales.

Zeolites represent an appropriate class of solid surface material to examine such specificity. Naturally occurring zeolites are microporous aluminosilicates whose porosity can reach 0.3 ml/g. The pore openings are normally from 3 to 8 Å in diameter, and form uni-, two-, or three-dimensional pore networks depending on zeolite type.

Although a pure silicon oxide zeolite would be neutral in charge, all natural zeolites contain a high amount of aluminium, giving rise to a charge deficiency in the lattice that is compensated by exchangeable cations located in the pores. Many zeolites are prepared synthetically, as are zeolite analogues such as aluminophosphates.

The abundance of new zeolite structures that have been synthesised suggests an ever-increasing variety of zeolites will become available for future analyses. In addition, zeolites are known to be well-tolerated by microorganisms and zeolites are normally stable both in wet and dry states, rendering them compatible with genetic and biochemical analyses.

S. Munsch et al. (2001) describes the use of zeolites for the isolation of amino acids. It is disclosed that the amino acids are located inside the pores of the zeolite structure and that the adsorption and desorption is determined by change in pH or by hydrophobicity.

As described above improved methods for immobilisation of a protein onto solid surfaces are potentially useful for optimising the activity and the efficiency of the protein. Thus, it is a requirement for such an immobilisation that a polypeptide tag is directed to a site opposite to the active site of the protein whereby the orientation of the enzyme or protein is predetermined in order to expose the active site.

Accordingly, isolation and characterisation of proteins able to distinguish the crystallographic planes of solid surfaces is disclosed herein and the proteins associated or attached with the polypeptide tag of the invention described herein bound to their cognate solid surfaces such as zeolite with high affinity and permitted enzyme immobilisation without loss of specific activity.

### SUMMARY OF THE INVENTION

Accordingly, the present invention provides polypeptide tags capable of binding specifically to a microporous material, where the microporous material is selected from the group consisting of zeolite or similar solid surfaces. These polypeptide tags are useful for the immobilisation of proteins to a solid surface whereby loss of activity of said protein is negligible.

The method to be used for associating or attaching the polypeptide tag with the protein is either by recombinant expression of the protein tag or by chemical treatment. In both cases the protein tag is attached to a protein of a position which favours the exposure of the active site of the protein towards the solvent whereby a high activity of the protein is maintained.

The advantage of immobilising the protein, e.g. an enzyme, on a solid surface prior to introducing it to a reaction is that the subsequent removal of the enzyme is very easy and may be performed either by decanting, filtration, sedimentation or centrifugation. Subsequently, the immobilised enzymes may be reused in further substrates.

### DETAILED DISCLOSURE OF THE INVENTION

Thus, in the broadest aspect of the present invention, there is provided a polypeptide tag and a method of using the polypeptide tag sequence for immobilising a protein on a solid surface whereby loss of activity of said protein is less than 10% of the initial activity prior to immobilisation, the method comprising the steps of:

1. selecting a polypeptide tag capable of binding to the surface,

2. immobilising said protein by the steps of:
attaching said polypeptide tag to the protein, and
binding said polypeptide tag to the solid surface
   where step (a) and (b) is performed simultaneously or sequentially and when performed sequentially, the order of step (a) and (b) is random, subject to the limitation that the polypeptide tag does not consist only of histidine residues.

As described above, there are at least two alternative ways of immobilising the protein to the solid surface. The first way is by attaching the polypeptide tag to the protein followed by binding of the polypeptide tag to the solid surface whereby the protein becomes immobilised. The second was is by binding the polypeptide tag to the solid surface followed by attachment of the bound polypeptide tag to the protein.

Binding/Immobilisation

In the present context the term “binding” relates to the association provided between the polypeptide tag and the solid surface. In a preferred embodiment of the present invention the binding described in step 1 above is a specific binding of the polypeptide tag to the surface where the polypeptide tag recognises a specific region or plane on the solid phase.

In the present context, the term “specific binding” relates to the binding between molecules wherein one of the molecules has an area on its surface, or a cavity to which an other molecule binds specifically. In a preferred embodiment of the present invention the polypeptide tag has the ability to distinguish solid surfaces having identical atomic compositions and varying only in the spatial orientation of surface atoms.

Analyses of the binding between the polypeptide tag and the solid surface showed that repeating the polypeptide tag sequence provided several desirable features for protein engineering. In addition to retaining their binding properties when fused to or associated with a variety of other proteins, repeating polypeptide tags bound to their target solid surfaces with high affinity and specificity. Thus, polypeptide tags have been identified and characterised that are able to distinguish subtle differences between solid surfaces.

A population of repeating polypeptide tags may be expressed on the surface of a bacterium. The repeating polypeptide tags that bind to the solid surface cause bacteria that display the polypeptide tag repeats to bind to the solid surface. Bacteria bound to the solid surface may be recovered and transferred to bacteriological growth medium. The method used for recovering the bacteria bound to the solid surface can be distinguished from the large range of conventional isolation operations consisting of sedimentation, filtration, centrifugation and decanting.

Accordingly, when repeating the polypeptide tag the binding between the polypeptide tag and the solid surface is enhanced and becomes stronger. In the present context, the term “the binding is enhanced” relates to an increase in the binding strength between the polypeptide tag and the solid surface and subsequently it is more difficult to separate the polypeptide tag from the solid surface. I one embodiment of the present invention the enhancement of the binding provided by a repeating polypeptide tag relative to a single polypeptide tag is increased by at least 10%, such as at least 20%, e.g. at least 50%, such as at least 75%, e.g. at least 100%, such as at least 200%, e.g. at least 300%, such as at least 400%, e.g. at least 500%, such as at least 600%, e.g. at least 700%, such as at least 800%, e.g. at least 900%, such as at least 1000%.

Furthermore, when repeating the polypeptide tag the avidity between the polypeptide tag and the solid surface is enhanced. In the present context the term “avidity” relates to an increased preference of the solid surface and the polypeptide tag to bind together rather than performing binding to other molecules or compounds. In one embodiment of the present invention the enhancement of the avidity provided by a repeating polypeptide tag relative to a single polypeptide tag is increased by at least 10%, such as at least 20%, e.g. at least 50%, such as at least 75%, e.g. at least 100%, such as at least 200%, e.g. at least 300%, such as at least 400%, e.g. at least 500%, such as at least 600%, e.g. at least 700%, such as at least 800%, e.g. at least 900%, such as at least 1000%.

In a preferred embodiment of the present invention the polypeptide tag is repeated at least 2 times, such as at least 3 times, e.g. at least 4 times, such as at least 5 times, e.g. at least 6 times, such as at least 7 times, e.g. at least 8 times, such as at least 9 times, e.g. at least 10 times, such as at least 15 times, e.g. at least 20 times, such as at least 25 times, e.g. at least 30 times, such as at least 40 times, e.g. at least 50 times, such as at least 60 times, e.g. at least 70 times, such as at least 80 times, e.g. at least 90 times, such as 100 times.

When immobilising a polypeptide tag to a solid surface according to the present invention it is essential that the active site of the protein becomes exposed to the solvent. This immobilisation is accomplished by attaching the polypeptide tag to a site opposite of the active site. In the present context the term “immobilisation” relates to the act of limiting movement of the protein or making the protein incapable of movement. In one embodiment of the present invention the binding constant between the polypeptide tag and the solid surface is less than 1 nM, e.g. less than 0.5 nM, such as less than 0.1 nM, e.g. less than 0.05 nM, such as less than 0.01 nM, e.g. less than 0.005 nM such as less than 0.001 nM.

It could be assumed that the basic amino acids, lysine and arginine, should be highly represented within said polypeptide tag, as expected for proteins that would normally adhere to weakly acidic surfaces like some zeolites. Although most of the amino acids may contribute to the overall structure of the repeating polypeptide tag, it is likely that only a few amino acids constitute the binding site which contacts the recognised surface. In such a case, only a minor part of the amino acid sequence must be conserved and the overall amino acid composition is unlikely to vary from random in a statistically significant manner. Therefore, as expected for proteins where the relative position of the amino acid side chains rather than the overall composition of amino acids is critical for binding properties, the prevalence of arginine and lysine codons is not statistically significant when examined by Fisher's Exact Test.

Attachment

In order to immobilise the protein onto the solid surface the protein must be attached to or associated with the polypeptide tag. In the present context the term “attachment” is used interchangeably with “associated” are relates to any type or connection made between the polypeptide tag and the protein. This connection may e.g. be performed by recombinant DNA procedures or by chemical treatment.

In a preferred embodiment of the present invention, the approach for attaching the protein tag to the protein is by recombinant DNA procedures which allow the orientation of the protein on the solid surface to be controlled, in turn allowing the active site to be well-exposed to the solvent. It also allows the immobilised protein to flex freely, retaining full catalytic activity. In the case of a multisubunit protein, flexibility can be retained by detaching the polypeptide tag to only one or a small number of subunits. In addition, adding such a polypeptide tag by recombinant procedures simplifies the purification of the enzyme from crude extracts and can reduce the cost of manufacture.

In another embodiment of the present invention the approach of attaching the polypeptide tag onto the protein is by chemical treatment e.g. by disulphide bonding between SH-containing residues of the protein with SH-containing residues of the protein tag. Using this strategy the precise location of the attached polypeptide tag is more difficult to predetermine. Another chemical treatment that could be used in the present invention is the use of a linkage molecule such as e.g. cyanobromide.

Protein

When immobilising the protein onto the solid surface it is an object of the present invention that the immobilisation should be provided without loss of activity of the immobilised protein, relative to the initial activity prior to immobilisation. In the present context the term “initial activity prior to immobilisation” relates to the activity of the protein free in a solution and non-immobilised.

In one embodiment of the present invention, loss of activity of the immobilised protein relative to no-immobilised protein in solution is less than 10%, such as less that 5%, e.g. less than 1%, such as less than 0.5%.

In another embodiment of the present invention the attachment between the polypeptide tag element and the protein element is in the form of a fusion protein between the elements. In the present context the term “fusion protein” is used interchangeably with “hybrid protein” and relates to a protein created by expression of a hybrid gene, made by genetic engineering. Two separate gene sequences are combined, usually by cloning the appropriate cDNA into an expression vector and the protein expressed comprises the polypeptide tag and the protein.

In a further embodiment of the present invention the fusion protein is provided recombinantly. In the present context, the terms “recombinantly” or “recombinant” relate to genetic engineering, molecular biology technology or cloning, the collection of techniques that allow the purification, manipulation and use of genetic material. These techniques are mostly based on the use of enzymes purified from bacteria and animal cells, that allow steps such as DNA copying (replication), conversion into RNA (transcription), and into fusion protein (translation). These steps include specific cutting of DNA at defined sites, joining or splicing of DNA fragments, and numerous other conventional procedures which allow manipulation down to the level of a single base pair. These techniques allow the manipulation of DNA; the conversion of these genes into fusion protein by expression in biological vats such as bacteria, and yeast; the bioengineering of viruses; the production of engineered organisms such as transgenic animals and knockout mice; and the development of DNA-based forensic and diagnostic tests.

In one embodiment of the present invention the protein is a protein expressed on the surface of a cell. In yet an embodiment of the present invention the protein expressed on the surface of a cell provides a site for removal or immobilisation of at least one single cell type present in a solution.

In yet another embodiment the immobilised protein of the present invention is used as chromatography column material for the purification of an analyte. The types of chromatography where the immobilised protein can be applied include ion exchange chromatography, gel filtration, hydrophobic interaction chromatography, affinity chromatography, reversed phase chromatography and expanded bed adsorption.

A common problem in the use of protease, is that proteases themselves are sensitive to proteolytic digestion. In solution, where the proteases can contact each other, the net proteolytic activity declines with incubation. Attaching the protease to a solid surface allows it to cleave only those proteins that are free in solution, not the other protease molecules attached to the surface. Immobilisation of protease delays decline in their activity. In one preferred embodiment the immobilised protein of the present invention is used for hydrolysi of a substrate.

In a particular embodiment of the present invention the protein being immobilised is selected from the group consisting of an antibody, an antigen, a receptor, a hormone, a lectin, an enzyme and a protease.

Other types of specifically binding molecules, such as e.g. biotin, avidin and sugar, may also be immobilised onto the solid surface.

Surface

The solid surface to which the protein is being immobilised may be distinguished by having not only different atomic compositions also when only varying in the spatial orientation of surface atoms. In the present context the term “solid surface” is used interchangeble with the term “microporous material” and relates to a surface substantially not being dissolved in the liquid sample in which it is provided and/or where the crystal structure of the solid surface is substantially sustained in the solvent.

In the present invention the term “substantially not being dissolved” and/or “substantially sustained” relates to at the most 1% of the solid surface material being dissolved/sustained, such as at most 90%, e.g. at most 75%, such as at most 50%, e.g. at most 25%, such as at most 10%, e.g. at most 5%.

In a preferred embodiment of the present invention the surface comprises at least one aluminum moiety, at least one silicate moiety, and/or at least one phosphate moiety. The solid surface of the invention is preferably selected from the group consisting of meso- and microporous materials such as zeolite or similar solid surfaces.

In the present context the term “similar solid surfaces” relates to solid surfaces selected from the group consisting of intercalated hydrotalcites and intercalated clays, and other aluminum silicates, aluminum phosphates, clays, metal oxides hydrotalcites, oxide powders, activated carbon, mica, glass and quartz.

Preferably the solid surface is an aluminosilicate such as zeolite. Zeolites are a class of non-biological material to which the polypeptide tag of the present invention has shown to bind specifically. Zeolites are either naturally occurring or synthetically produced. Naturally occurring zeolites are microporous aluminosilicates forming uni-, two-, or three-dimensional pore networks depending on zeolite type. Although a pure silicon oxide zeolite would be neutral in charge, all natural zeolites contain a high amount of aluminum, giving rise to a charge deficiency in the lattice that is compensated by exchangeable cations located in the pores. Many zeolites are prepared synthetically, as are zeolite analogs such as aluminophosphates. The abundance of new zeolite structures that have been synthesised suggests an ever increasing variety of zeolites will become available for future analyses. In addition, zeolites are known to be well-tolerated by microorganisms and zeolites are normally stable both in wet and dry states, rendering them compatible with genetic and biochemical analyses.

In particular embodiments of the present invention the meso- and microporous material is selected from the group of zeolites consisting of AFI, EMT, FAU and MFI.

The meso- and microporous material may also be characterised by the porosity and/or the pore size. Thus, useful meso- and microporous material have a pore size in the range of 1-500 Å, such as 1-100 Å, e.g. 1-50 Å, such as 1-20 Å, e.g. 1-15 Å, such as 2-10 Å, e.g. 3-8 Å, such as 5-8 Å, e.g. 6-8 Å. The pore size is at the most 1000 Å, such as at the most 500 Å, e.g. at the most 250 Å, such as at the most 150 Å, e.g. at the most 100 Å, such as at the most 75 Å, e.g. at the most 50 Å, such as at the most 40 Å, e.g. at the most 30 Å, such as at the most 20 Å, e.g. at the most 10 Å, such as at the most 8 Å, e.g. at the most 6 Å, such as at the most 5 Å, e.g. at the most 3 Å.

In another embodiment of the present invention the meso- and microporous material has a porosity of at least 0.01 mL/g, such as at least 0.05 mL/g, at least 0.1 mL/g, such as at least 0.2 mL/g, at least 0.03 mL/g, such as at least 0.4 mL/g, at least 0.5 mL/g, such as at least 0.10 mL/g, at least 0.20 mL/g, such as at least 0.50 mL/g, at least 0.75 mL/g, such as at least 1.00 mL/g,

Polypeptide Tag

The inventors of the present invention found that polypeptide tags may have a high degree of specificity, i.e. binding one type of solid surface avidly while binding very weakly, if binding at all, to other solid surfaces. The repeating polypeptide tags bind avidly to certain solid surfaces such as aluminum silicate zeolites and very weakly to others solid surfaces as well as to other solid surfaces similar to zeolite. In fact, the polypeptide tags of the present invention are able to distinguish crystallographic planes of identical atomic composition solely on the basis of atomic orientation.

In the present context the term “polypeptide” relates to a sequence of at least 10 amino acid residues to form a chain of amino acids joined by peptide bonds. In the present context the term “polypeptide tag” relates to a polypeptide capable of recognising the solid surface to which the protein to which the polypeptide is attached, is being immobilised.

In one specific embodiment of the present invention the polypeptide tag comprises at least two lysine residues. In another preferred embodiment of the present invention the polypeptide tag does not consist only of histidine residues polypeptide tag, such as the histidine-tag.

The polypeptide tag as defined herein comprises at the most 500 amino acid residues, such as at the most 400 amino acid residues, e.g. 300 amino acid residues, such as at the most 200 amino acid residues, e.g. 100 amino acid residues, such as at the most 75 amino acid residues, e.g. 50 amino acid residues, such as at the most 25 amino acid residues, e.g. 20 amino acid residues, such as at the most 15 amino acid residues, e.g. at the most 10 amino acid residues. Preferably the polypeptide tag comprises at the most 14 amino acid residues or at the most 21 amino acid residues.

In a preferred embodiment of the present invention the polypeptide tag has at least 30% amino acid sequence identity to SEQ ID NO 1, such as at least 40%, e.g. at least 50%, such as at least 60% e.g. at least 70%, such as at least 80%, e.g. at least 85%, such as at least 90%, e.g. at least 95%, e.g. at least 97%, such as at least 98%, e.g. at least 99%, such as at least 100%.

In a preferred embodiment of the present invention the polypeptide tag has at least 30% amino acid sequence identity to SEQ ID NO 2, such as at least 40%, e.g. at least 50%, such as at least 60% e.g. at least 70%, such as at least 80%, e.g. at least 85%, such as at least 90%, e.g. at least 95%, e.g. at least 97%, such as at least 98%, e.g. at least 99%, such as at least 100%.

As mentioned above the numbers of amino acid residues building up the polypeptide tag only account for a polypeptide tag without any repeats encounted. Thus, the number of amino acid residues and the number of repeats must be multiplied in order to obtain the total number of amino acid residues present in the entire polypeptide tag including the repeats. The actual total number of amino acid residues may deviate from the calculated number due to insertion or deletion of amino acid residues within each single repeat. Thus, In accordance with the invention the number of amino acid residues within each repeat deviates by at the most 15 amino acid residues, such as at most 10 amino acid residues, e.g. at most 5 amino acid residues, such as at most 4 amino acid residues, e.g. at most 3 amino acid residues, such as at most 2 amino acid residues, e.g. at most 1 amino acid residues.

In another embodiment of the present invention the amino acid sequence identity between the repeating polypeptide tags is at least 30, such as at least 40%, e.g. at least 50%, such as at least 60% e.g. at least 70%, such as at least 80%, e.g. at least 85%, such as at least 90%, e.g. at least 95%, e.g. at least 97%, such as at least 98%, e.g. at least 99%, such as at least 100%.

In a further useful embodiment of the present invention the polypeptide tag is capable of controlling the orientation of proteins immobilised onto a solid surface, whereby the active site of the protein become exposed to the solvent such that the loss of activity relative to non-immobilised protein is negligible.

In some cases the polypeptide tag is immobilised on to a protein having multible subunits. In a preferred embodiment of the present invention the polypeptide tag is provided on at least one subunit of a protein.

### Further Embodiments

In a further aspect the invention pertains to a method for isolating an analyte from a liquid sample is provided, the method comprising the steps of:
1. selecting a protein immobilised according to the present invention, said protein is capable of specifically binding to the analyte,
2. contacting said immobilised protein with the liquid sample,
3. permitting said immobilised protein to react with the analyte to obtain a complex of the immobilised protein and the analyte,
4. optionally washing said complex, and
5. eluting the analyte from said complex.

In the present context “a liquid sample” relates to any sample found in the form of liquid or solid or gas which is liquefied at the time of assaying. The liquid sample is typically selected from the group consisting of a fermentation medium, wastewater, blood, milk and urine, diary products and/or a chemical reaction.

Accordingly, in yet a further aspect of the present invention there is provided a chromatography column material for the purification of an analyte. The analytes that can be purified using the chromatography material of the invention includes as examples proteins, haptens, immunoglobulins, antibodies, hormones, polynucleotides, steroids, drugs, and infectious disease agents such as bacteria or viruses.

A common problem with the use of enzymes as catalysts is that an enzyme used to catalyse one step in a process may interfere with a subsequent step. In these instances, the first enzyme must be inactivated or removed. For example, restriction enzymes used to digest DNA for gene cloning must be removed prior to ligating the DNA fragments to each other. Another common method in recombinant DNA technology is the removal of 5-phosphates from the ends of a piece of DNA to force it to ligate to DNA fragments retaining their 5′phosphates. Alkaline phosphatases are generally used to remove 5′phosphates. However, the alkaline phosphatase must be removed before introduction of the DNA retaining its 5′phosphates.

In a further embodiment of the present invention the immobilised protein is reused. Thus, after use of an immobilised enzyme for the proteolytic digestion of a molecule the immobilised enzyme is recovered from the liquid sample and subsequently reused in another proteolytic digestion at a later stage or in a different medium. In an embodiment of the present invention the immobilised protein is used for the hydrolysis of a molecule.

One object of the present invention is to provide a cell comprising a surface molecule comprising the polypeptide tag. Preferably, the polypeptide is described as disclosed herein.

In still a further aspect, the present invention provides a material having at least one surface onto which a polypeptide tag has been bound is provided, said polypeptide tag having at least 30% identity to SEQ ID NO. 1 or SEQ ID NO. 2, such as at least 40%, e.g. at least 50%, such as at least 60% e.g. at least 70%, such as at least 80%, e.g. at least 85%, such as at least 90%, e.g. at least 95%, e.g. at least 97%, such as at least 98%, e.g. at least 99%, such as at least 100%. In another embodiment of the present invention the material to which the polypeptide tag is bound includes meso- and microporous materials as described earlier.

In yet another aspect the present invention relates to a fusion protein having a polypeptide tag bound thereto, said polypeptide tag having at least 30% identity to SEQ ID NO. 1 or SEQ ID NO. 2, such as at least 40%, e.g. at least 50%, such as at least 60% e.g. at least 70%, such as at least 80%, e.g. at least 85%, such as at least 90%, e.g. at least 95%, e.g. at least 97%, such as at least 98%, e.g. at least 99%, such as at least 100%.

The following non-limiting examples and drawings will further illustrate the invention.

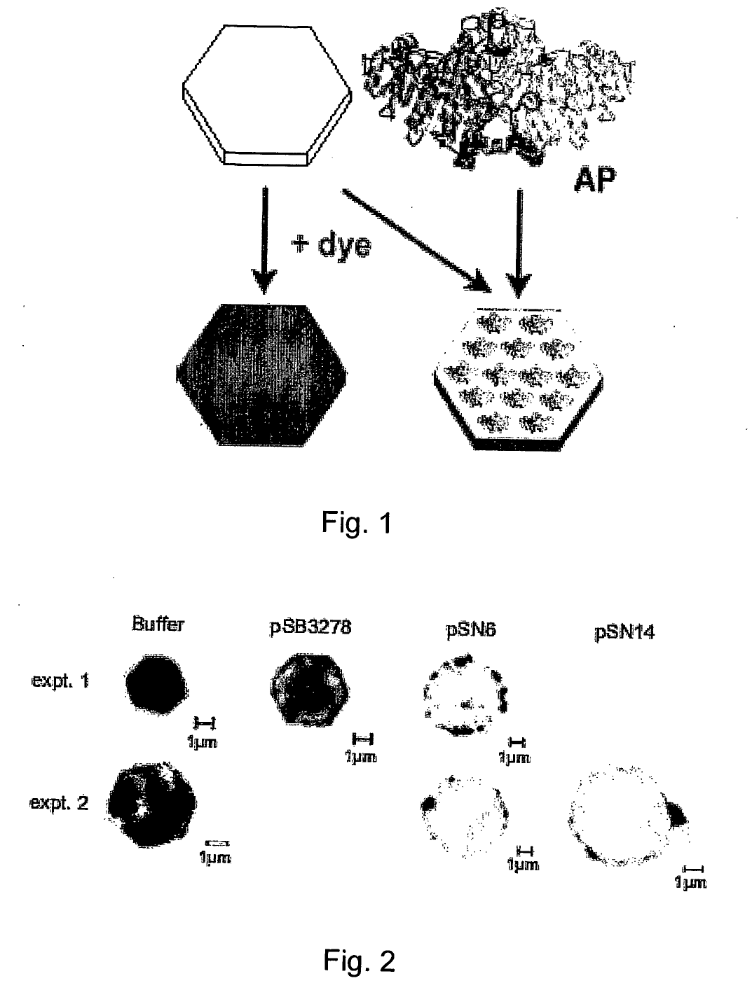

FIG. 1 represents a dye exclusion assay of proteins binding to EMT. The figure summarises the experiment and shows features of AP. Components are not drawn to scale. alkaline phosphatase (AP) is a dimer of two identical subunits with an overall length of approximately 10 nm represented as a ribbon diagram in the upper right of the panel. The first 6 amino acids of alkaline phosphatase replaced by the repeating polypeptide tags are represented by the space-filling models attached to the base of each subunit. Each subunit contains a catalytic site. A phosphate in the catalytic site of the right subunit can be seen as a space-filling model near the top of the subunit. The zeolite was first incubated with the alkaline phosphatase preparations or with buffer lacking any protein and then incubated with the dye, TMR-PLL. Interference with staining of the (001) face by alkaline phosphatase is depicted. The experiment only examines the surfaces of EMT recognised by he APs, not the relative orientations of the alkaline phosphatase molecules to each other. FIG. 2 represents dye exclusion by proteins binding to EMT. The experiment is outlined in FIG. 1. Fluorescence micrographs were obtained with the indicated sources of AP. Negative images of the fluorescent channel are shown.

### EXAMPLES

Methods and Materials

Strains, Plasmids and Libraries:

The repeating polypeptide tag library was a pool of all libraries described in S. Brown (1997) and S. Brown et al. (2000). The phoA expression vector, pSB2991 and the ΔphoA host strain, S2157, were described previously by S. Brown (1997). pSB3278 was described previously by S. Brown et al. (2000) and encodes the RP6/1′ form of alkaline phosphatase.

Description of Zeolites:

The four microporous crystalline zeolites were used in the present study are shown in table 1 below. Three synthetic zeolites; ZSM-5 (MFI), Faujasite (FAU) and EMT-zeolite (EMT), and one synthetic aluminum phosphate: AlPO₄-5 (AFI). The FAU, AFI and EMT were synthesized in SINTEF, whereas the MFI was obtained from Vereinigte Aluminiumswerke, Germany. The samples used here were chosen due to their apparently well-defined solid surfaces. The FAU crystals are octahedral in outline, reflecting cubic symmetry, with the eight (111) faces developed, so all external faces are equivalent. On these faces, the pore openings are distributed in a hexagonal pattern, with a distance of about 17 Å between adjacent pores (center-center). EMT is structurally related to FAU, but with hexagonal symmetry. The EMT sample has crystals with hexagonal planar outline with its basal (001) faces essentially equivalent to the (111) faces of FAU, whereas it has six equivalent side faces parallel to its c-axis. On these side faces the pore openings are oval in shape. The AFI is also hexagonal with its basal (001) faces similar to the (111) faces of FAU, but with a smaller distance of approximately 14 Å between adjacent pore openings. It's six side faces are equivalent and lack pore openings, as the pores are uni-dimensional along the c-axis. Finally, MFI has orthorhombic symmetry, with several different faces exposed, but all with smaller pore openings than the other materials (Table 1).

Enrichment and Manipulation of DNA:

Enrichment for zeolite-binding polypeptide tag attached proteins was conducted and monitored as described by S. Brown (1997). Zeolites were washed in sterile 10 mM potassium phosphate, pH 7.0, 0.1 M KCl and neutral pH verified before exposure to bacteria. The repeating oligonucleotides were transferred to pSB2991 by XhoI/PstI.

Transformants of S2157 were tested for production of recombinant proteins by SDS gel electrophoresis of periplasmic extracts.

Measurement of Binding:

Steady-state labeling of protein with ³⁵SO₄ and preparation of periplasmic extracts was conducted also as described by S. Brown (1997). Binding measurements were conducted in 1 ml 10 mM potassium phosphate, pH 7.0, 0.1 M KCl, 1% Triton X-100 (PKT) containing 1 mg indicated zeolite. Zeolites were washed in PKT and neutral pH verified before use. Extracts and the zeolites were incubated with constant gentle agitation for 20 minutes at room temperature. The zeolites with any adhering protein were recovered by centrifugation and analyzed by SDS gel electrophoresis.

Fluorescence Microscopy:

Tetramethylrhodamine-poly-L-lysine was prepared by reacting NHS-tetramethyl-rhodamine (Molecular Probes) with poly-L-lysine (molecular weight 150,000-300,000, Sigma). Coupling was conducted at a ratio of 1:50 of NHS-TMR relative to lysine residues and the product purified by size-exclusion chromatography. Most of the dye eluted with high molecular weight material.

Polypeptide tag attached proteins were purified from transformants of S2157 by method through DEAE chromatography and dialyzed against 10 mM potassium phosphate, pH 7.0, 0.1 M KCl. Dialysates were made 1% Triton X-100 before use. EMT was washed with PKT buffer and incubated with the polypeptide tag attached proteins 20 minutes at room temperature with gentle agitation. A solution of TMR-PLL in PKT buffer was added directly to the polypeptide tag attached protein-EMT suspension and incubated at least 10 minutes at room temperature with gentle agitation. Suspensions were examined by laser scanning confocal microscopy.

Alkaline Phosphatase Immobilization:

Bacteria were grown and periplasmic extracts prepared as described by S. Brown (2000). Extracts were incubated for 1 hour with gentle agitation at room temperature with zeolites washed and resuspended in either PKT or 10 mM Tris-HCl pH 7.6, 0.1 M NaCl, 1% Triton X-100. Zeolites were recovered by centrifugation, washed and resuspended in 0.1 M Tris-HCl pH 8.0. Alkaline phosphatase activity was measured in 0.1 M Tris-HCl pH 8.0 containing 1 mM 4-nitrophenyl phosphate at room temperature and absorption at 410 nm measured after addition of KH₂PO₄. Samples representing equal amounts of enzyme activity were electrophoresed on SDS gels and stained with Coomassie Brilliant Blue. Protein was quantified by scanning.

### Example 1

Studying of EMT-Binding Polypeptide Tag

The object of this experiment is to examine the candidate EMT-binding polypeptide tag attached proteins as soluble proteins, the DNA encoding the repeating polypeptide tags was transferred from lamb, the gene encoding the bacterial surface protein, to phoA, the gene for alkaline phosphatase.

Three candidates producing abundant hybrid proteins were isolated and examined further. Two of them adhered avidly to EMT, the products of pSN6 (SEQ ID NO. 1 having 4 repeats) and pSN14 (SEQ ID NO. 2 having 7 repeats), AP_{pSN6} and AP_{pSN14}, respectively. The most detailed characterisation was conducted on AP_{pSN6}, but AP_{pSN14} behaved similarly.

### Example 2

The ability of AP_{pSN6}, AP_{pSN14} and a control alkaline phosphatase lacking a repeating polypeptide tag, AP_{pSB2991} (SEQ ID NO. 3), to adhere to a panel of zeolites was measured using radiolabelled proteins. The panel of zeolites comprised four materials selected to represent a variation in charge density, pore size and pore pattern on the crystal solid surfaces is seen in Table 1. In addition, on each material, different crystal solid surfaces with different pore opening patterns were exposed. The difference in charge density generates differences in the secondary property, hydrophobicity. The lower the charge, the more hydrophobic the material.

TABLE 1
Some key properties of the microporous materials studied.

| | Charge/ | Pore size^{[a]} | Pore/ | |
|---|---|---|---|---|
| Material | density | (Å) | geometry | Composition |
| AFI | very low | 7.3 | 1D | Aluminophosphat |
| EMT | high | 7.3/7.6 × 6.5^{[b]} | 3D | Aluminosilicate |
| FAU | high | 7.4 | 3D | Aluminosilicate |
| MFI | low | 5.3 × 5.6/ | 3D | Aluminosilicate |
| | | 5.1 × 5.5 | | |

^{[a]}Narrowest diameter of pore openings. EMT and MFI have two different sets of pores.
^{[b]}The 7.3 Å pores are those on the (001)-surface and the 7.5 × 6.5 Å pores are those on the (100) and (010) faces of EMT.

It was not possible to detect binding by AP_{pSB2991} to any of the tested zeolites. The failure of AP_{pSB2991} to adhere to zeolites is not surprising considering alkaline phosphatase is acidic. Both AP_{pSN6} and AP_{pSN14} were able to bind avidly to EMT and FAU, but very weakly to the zeolites with lower charge densities, MFI and AFI. The dissociation constant of AP_{pSN6} from EMT was measured and found to be less than 100 pM.

The binding behaviour of AP_{pSN6} and AP_{pSN14} to the panel of zeolites suggests two hypotheses regarding the recognised zeolite solid surface feature. The first hypothesis is the proteins bound more avidly to the more highly charged zeolite surfaces. Alternatively, the polypeptide tag attached proteins recognised a more subtle feature common to the exposed surfaces of EMT and FAU. The (111) face of FAU is identical to the (001) face of EMT apart from the pore-openings of EMT being 1-2% smaller than those of FAU. Neither MFI nor AFI have exposed surfaces with atomic orientations similar to the (001) face of EMT. The second hypothesis is AP_{pSN6} and AP_{pSN14} recognised and adhered to a feature unique to the (001) face of EMT.

EMT forms thin, flat hexagonal crystals and the broad hexagonal face is the (001) face. The side faces of EMT are the (100) and (010) faces which are identical and all exposed surfaces of EMT have similar charge densities. Since the crystallographic faces of EMT are readily distinguished by light microscopy, we tested which faces attracted AP_{pSN6} and AP_{pSN14}. The test we used was a dye-exclusion assay. A basic dye, tetramethylrhodamine-poly-L-lysine (TMR-PLL) is expected to adhere to all zeolite faces by electrostatic attraction. The experimental strategy is shown in FIG. 1. It can be seen in FIG. 2 (buffer) that TMR-PLL adhered to the broad hexagonal (001) face of EMT. The extensive binding by TMR-PLL to the (001) face of EMT prevented observing whether it adhered to the other faces of EMT. When the EMT is first incubated with a candidate binding polypeptide tag attached protein, that protein may obscure faces of the zeolite and later prevent binding by TMR-PLL (FIG. 1). If AP_{pSN6} and AP_{pSN14} adhere only to the (001) face of EMT, they will prevent TMR-PLL from binding only on the broad, hexagonal face while permitting TMR-PLL to bind to the thin edges.

It can be seen in FIG. 2 that both AP_{pSN6} and AP_{pSN14} prevented staining of the broad, hexagonal face while permitting staining of the edges of the EMT crystals. Thus, it was not solely the surface charge density of the zeolites which caused AP_{pSN6} and AP_{pSN14} to distinguish the EMT and FAU from MFI and AFI.

As a control protein, an alkaline phosphatase similar to AP_{pSN6} and AP_{pSN14} but with an apparently unrelated function, AP_{pSB3278} (SEQ ID NO. 4) was chosen. AP_{pSB3278} alters the shape of growing gold crystals and was chosen because its predicted isoelectric point is the same as AP_{pSN6}.

A control protein with the same isoelectric point permitted the inventors of the present invention to ask two questions. Is the inhibition of staining of the (001) face solely due to the charge of the added polypeptide tag attached protein? If so, AP_{pSB3278} will also exclude dye from the (001) face. Secondly, since the polypeptide tag attached proteins all have similar isoelectric points, they can all be purified by the same protocol and thus will have the same contaminants. This addresses the question, is the inhibiting entity AP_{pSN6} or AP_{pSN14}, or is the inhibiting entity a contaminant of the protein preparations? It can be seen in FIG. 2 that AP_{pSB3278} permitted TMR-PLL to stain the (001) face of EMT.

Thus, both questions were answered. AP_{pSN6} and AP_{pSN14} and not a contaminant excluded dye from the (001) face of EMT. The quality of AP_{pSN6} and AP_{pSN14} necessary for recognition of the (001) face of EMT is not solely the basic nature of the polypeptide tag attached proteins. Furthermore, since the broken bonds on all of the EMT surfaces exposed to water are likely to terminate with hydroxyl groups, the recognised feature of EMT is probably the precise spatial orientation of the surface atoms.

### Example 3

Determination of the Protein Orientation

The activity of an immobilised enzyme is, in part, determined by its orientation. In an ideal situation, the immobilised enzyme would have its catalytic site well-exposed to the solvent. The catalytic site of alkaline phosphatase is on the opposite face from the attachment of the repeating polypeptide tags. Therefore, if alkaline phosphatase is bound to a solid support by the repeating polypeptide tags, the catalytic site will face the solvent.

The immobilised behaviour of AP_{pSN6} was examined in two independent experiments. In both, protein extracts were incubated with the zeolite powders and washed the powders extensively. The enzyme activity was then measured and samples representing equal amounts of enzyme activity were examined by SDS gel electrophoresis. The amount of enzyme was then quantified by staining and scanning the gel. In the first experiment enzymes was adsorbed in the phosphate buffer used in the binding assays, PKT. 99% of the enzyme were found associated with the FAU-AP_{pSN6} relative to the same number of enzyme units associated with the soluble AP_{pSN6}. Thus, AP_{pSN6} retained all of its activity when adsorbed on FAU. EMT-AP_{pSN6} behaved similarly to FAU-AP_{pSN6}. Since phosphate is an inhibitor of alkaline phosphatase, the experiment was repeated adsorbing the enzyme in Tris-HCl buffer. Here the inventors of the present invention found 87% the enzyme associated with the FAU-AP_{pSN6} relative to the same number of enzyme units associated with the soluble AP_{pSN6}. Thus, in the second experiment, AP_{pSN6} had slightly greater activity when bound to FAU than when in solution. The amount of enzyme associated with FAU after the enzyme assay was examined and found 94% of the enzyme remained associated. On the other hand, enzyme immobilisation on meso-porous silicates is often accompanied by reduction in activity and deterioration of kinetic parameters.

Conclusion

Zeolite chemistry and genetic selections provide a powerful combination to investigate the abilities of proteins and polypeptide tags to adhere to and distinguish solid surfaces. The inventors of the present invention used genetic selection to isolate repeating polypeptide tags that as hybrid proteins with an E. coli surface protein to provide a fusion protein caused the bacteria to adhere to an EMT zeolite. The EMT-binding property of the repeating polypeptide tags was retained as hybrid proteins with alkaline phosphatase. Measurements of binding to a panel of zeolites allowed us to infer the molecular features and thus the crystallographic plane recognised by the binding proteins. Both EMT-binding proteins excluded dye from the (001) face of EMT while permitting dye to adhere to the other crystallographic faces. Since the crystallographic faces of EMT differ only in the relative orientations of the constituent atoms and not in the relative abundance of the constituent atoms, the dye-exclusion experiment showed AP_{pSN6} and AP_{pSN14} recognised the relative orientations of the surface atoms per se. Thus, proteins able to distinguish differences between inorganic solid surfaces as subtle as the differences recognised between biological surfaces can be readily isolated. Based on the structure of alkaline phosphatase, the hybrid protein AP_{pSN6} was expected to bind to zeolites with its catalytic site facing away from the zeolite. As expected, AP_{pSN6} retained full enzymatic activity when immobilised on zeolite supports.

Thus, the inventors of the present invention suggest the strategy instituted here is general and can be used to isolate proteins able to adhere to many inorganic surfaces.

### Example 4

The aim of this example is to remove lactose from diary products, such as milk, using ZSM-5, ZSM-11, EU-1, ZSM-23, ZSM-57 and NU-87 as solid surfaces.

Lactose-free milk is prepared by incubation of the milk with beta-galactosidase. Beta-galactosidase hydrolyzes lactose, a disaccharide, into it's component monosaccharides, glucose and galactose. A variety of zeolites can be expected to absorb the monosaccharide products but not the disaccharide. These zeolites include ZSM-5, ZSM-11, EU-1, ZSM-23, ZSM-57 and NU-87. The modified beta-galactosidase would be bound to the selected zeolite and the coated zeolite mixed with milk. The close association with the zeolite would remove the products of lactose hydrolysis from solution. This removal of products drives the reaction to a greater extent thus improving the removal of lactose.

### Example 5

The aim of this example is to remove alkaline phosphatase from a solution.

For the immobilization of alkaline phosphatase use of a solid surface capable of subsequently removing released phosphate from solution. Alkaline phosphatase is commonly used to remove a phosphate from a biological molecule. The desired product is the dephosphorylated biological molecule and the released phosphate is later removed. If the released phosphate were immediately removed from the reaction, the reaction would be driven. The solid surfaces used for this purpose include type A, Beta, X, Y, ZSM-5, mordenite, climoptolite and gismondine zeolites, hydrotalcites, gibbsite (an aluminum hydroxide), and clays such as kaoline and bentonite, and mesoporous materials such as MCM-41 and MCM-48.

Sequences

| | | |
|---|---|---|
| SEQ ID NO. 1: | VKTQATSREEPPRLPSKHRPG | |
| | pSN6: | (VKTQATSREEPPRLPSKHRPG)₄ |
| SEQ ID NO. 2: | MDHGKYRQKQATPG | |
| | pSN14: | (MDHGKYRQKQATPG)₇ |
| SEQ ID NO. 3: | pSB2991; | QA |
| SEQ ID NO. 4: | pSB3278; | (QATSGSERMGHQSGTVHPGKT)₇ |
| | | (SKTSLGQSGASLQGSEKLTNG)₅ |

### REFERENCES

1. S. Munsch et al., “Adsorption and separation of amino acids from aqueous solutions on zeolites”, Chem. Commun. (Camb) October 7;(19):1978-9 (2001).

2. S. Brown et al., J. Mol. Biol, (299):725 (2000)

3. S. Brown, “Metal-recognition by repeating polypeptides”, Nature Biotechnology, March (15):269-272 (1997).

4. S. Brown, “Engineered iron oxide-adhesion mutants of the Escherichia coli phage λ receptor”, Proc. Natl. Acad. Sci. USA, September (89):8651-8655 (1992).

5. S. P. Graether et al., “β-Helix structure and ice-binding properties of a hyperactive antifreeze protein from an insect”, Nature, July 20 (406):325-328 (2000).

6. C. A. Knight et al., “Adsorption of -helical antifreeze peptides on specific ice crystal surface planes”, Biophysical Journal, February (59):409-418 (1991).

1. A method for immobilizing a protein on a microporous material, said microporous material is selected from the group consisting of zeolite or a similar solid surface whereby loss of activity of said protein is less than 10% of the initial activity prior to immobilizing, comprising the steps of:
(i) selecting a polypeptide tag capable of binding to the surface,
(ii) immobilizing said protein by the steps of:
(a) attaching said polypeptide tag to the protein, and
(b) binding said polypeptide tag to the solid surface
wherein step (a) and (b) are performed simultaneously or sequentially and when performed sequentially, the order of step (a) and (b) is random, further wherein the polypeptide tag does not consist only of histidine residues.

2. The method according to claim 1 wherein the binding in step (i) is a specifically binding of the polypeptide tag to the surface.

3. The method according to claim 1 wherein the polypeptide tag comprises at least two lysine residues.

4. The method according to claim 1 wherein the polypeptide tag comprises at the most 21-500 of amino acid residues.

5. The method according to claim 1 wherein said polypeptide tag has at least 30-100% amino acid sequence identity to SEQ ID NO 1.

6. The method according to claim 1 wherein said polypeptide tag has at least 30-100% amino acid sequence identity to SEQ ID NO 2.

7. The method according to claim 1 wherein the binding in step (i) is enhanced by repeating said polypeptide tag at least 2, 3, 4, 7, 10, 50, or 100 times.

8. The method according to claim 1 wherein the avidity of the polypeptide tag for the surface is enhanced by repeating said polypeptide tag at least 2, 3, 4, 7, 10, 50, 100 times.

9. The method according to claim 7 wherein the amino acid sequence identity between the repeating polypeptide sequences is at least 30-100%.

10. The method according to claim 1 wherein the protein is a protein expressed on the surface of a cell.

11. The method according to claim 1 wherein said attachment of the polypeptide tag to the protein provides a fusion protein.

12. The method according to claim 11 wherein said fusion protein is recombinantly provided.

13. The method according to claim 1 wherein the polypeptide tag is attached to the protein by chemical treatment.

14. The method according to claim 1 wherein the surface comprises at least one aluminum moiety, at least one silicate moiety and/or at least one phosphate moiety.

15. The method according to claim 1 wherein the similar solid surface is selected from the group consisting of meso- and microporous materials including hydrotalcite, clay, aluminosilicate, oxide powders, activated carbon, mica, glass, clinoptolite, gismondine zeolite, alluminate and quartz.

16. The method according to claim 15 wherein the zeolite is either naturally occurring or synthetically produced.

17. The method according to claim 15 wherein the meso- and microporous material is selected from the group of zeolites consisting of AFI, EMT, FAU and MFI.

18. The method according to claim 15 wherein the zeolite has a pore size in the range selected from the group consisting of 1-50 Å, 1-40 Å, 1-30 Å, 1-20 Å, 1-15 Å, 2-10 Å, e.g. 3-8 Å, 5-8 Å, 6-8 Å.

19. The method according to claim 1 wherein the protein is selected from the group consisting of an antibody, an antigen, a receptor, a biotin, an avidin, a hormone, a lectin, a sugar, an enzyme and a protease.

20. The method according to claim 1 wherein the polypeptide tag is bound directly to the solid surface.

21. A polypeptide tag that is capable of controlling the orientation of proteins immobilized on a microporous material, wherein said microporous material is selected from the group consisting of zeolite and similar solid surfaces.

22. The polypeptide tag according to claim 21 wherein the polypeptide tag comprises at least two lysine residues.

23. The polypeptide tag according to claim 21 wherein the polypeptide tag comprises at the most 21-500 amino acid residues.

24. The polypeptide tag according to claim 21 wherein the polypeptide tag is provided on at least one subunit of a protein.

25. The polypeptide tag according to claim 21 wherein said polypeptide tag has at least 30-100% amino acid sequence identity to SEQ ID NO 1.

26. The polypeptide tag according to claim 21 wherein said polypeptide tag has at least 30-100% amino acid sequence identity to SEQ ID NO 2.

27. A method for isolating an analyte from a liquid sample, said method comprising:
(i) selecting a protein immobilized according to the method of claim 1, wherein said protein is capable of specifically binding to the analyte,
(ii) contacting said immobilized protein with the liquid sample,
(iii) permitting said immobilized protein to react with the analyte to obtain a complex of the immobilized protein and the analyte,
(iv) optionally washing said complex, and
(v) eluting the analyte from said complex.

28. The method according to claim 27 wherein the liquid sample is selected from the group consisting of fermentation medium, wastewater, blood, milk, urine, dairy products and a chemical reaction.

29. The method according to claim 27 wherein the immobilized protein is reused.

30. A method of purifying analyte comprising contacting said analyte with a column chromatography material comprising a protein immobilized using the method of claim 1.

31. Use A method of hydrolyzing a molecule comprising contacting said molecule with a protein immobilized using the method of claim 1.

32. The cell comprising a surface molecule comprising the polypeptide tag according to claim 21.

33. A material having at least one surface onto which a polypeptide tag has been bound, wherein said polypeptide tag has at least 30-100% identity to SEQ ID NO.1 or SEQ ID NO. 2.

34. The material according to claim 33 wherein the surface is selected from the group consisting of meso- and microporous materials including zeolite, hydrotalcite, clay, aluminosilicate, oxide powders, activated carbon, mica, glass, clinoptolite, gismondine zeolite, alluminate and quartz.

35. A fusion protein bound to a polypeptide tag, wherein said polypeptide tag has at least 30-100% identity to SEQ ID NO. 1 or SEQ ID NO. 2.
